(19) United States
(12) Patent Application Publication
(10) Pub. No.: US 2020/0054588 A1
(43) Pub. Date: Feb. 20, 2020

(54) PHARMACEUTICAL PAIN RELIEF COMPOSITION

(71) Applicant: PROMINDUS SARL, Casablanca (MA)

(72) Inventors: Jean Jacques SERRANO, Montpellier (FR); Mohamed CHIBANE, Casablanca (MA)

(73) Assignee: PROMINDUS SARL, Casablanca (MA)

(21) Appl. No.: 16/471,421

(22) PCT Filed: Dec. 20, 2017
(86) PCT No.: PCT/IB2017/058193

§ 371 Date: Sep. 4, 2019

(30) Foreign Application Priority Data
Dec. 20, 2016 (FR) 16/62852

(51) Int. Cl. A61K 31/192; A61K 31/05; A61K 9/46; A61K 9/00; A61K 47/12; A61P 29/00; A61P 1/06

(52) U.S. Cl. CPC A61K 31/192; A61K 31/05; A61K 9/0007; A61P 1/06; A61K 47/12; A61P 29/00; A61K 9/0053

(57) ABSTRACT

The present invention thus relates to an anhydrous pharmaceutical composition comprising an anti-inflammatory principle chosen from the derivatives of aryl-propionic acid, an antispasmodic in the triphenol family and an effervescent excipient.

The present invention relates to a pharmaceutical preparation intended for treating acute pains requiring rapid action, ensuring long-term stability of the active principles contained, under testing environmental conditions, and very practical in transport and use.

Acute pains such as renal colic, colic or dysmenorrhea, in particular secondary dysmenorrhea caused by an underlying gynaecological problem, such as endometriosis, constitute pains that can be grouped together under the generic term inflammatory spasms.

In particular, renal colic designates a pain due to an obstruction of the urinary tracts. It manifests as an acute pain experienced suddenly in the lumbar region. It is due to an abrupt increase in pressure of the urine that can no longer flow. A renal colic crisis is thus spoken of.

Renal colics are due to an obstacle, in general a stone, in the urinary tracts that prevents the flow of urine and the causes of which may be highly diverse, such as inflammation of the urethra, a tumour in the renal tracts, fibrosis of the region, etc.

The pain manifests itself abruptly in the lumbar region, usually in the morning and/or during the night. It is felt on only one side, at the kidney affected. It may extend from the back as far as the side and towards the belly or the groin, and typically this pain irradiates towards the external genital organs.

Lithic renal colic (due to a urinary stone) represents 75% to 85% of the cases of renal colic.

The pain is mainly due to an increase in the pressure in the excretory tracts that is due to two factors. First of all, an oedema generated in contact with the stone cause an effect of retention of urine and will block the stone even more. Next, there is a stimulation of the local synthesis of prostaglandin E2 that will cause an increase in the blood flow and the glomerular pressure. The production of urine will further increase the pressure which in its turn will increase the production of prostaglandin E2. The smooth muscle will contract in order to expel the stone, which cannot move and the muscular fibres develop a veritable spasm.

The pain is variable in intensity but experiences particularly acute peaks. A dull pain frequently persists between each crisis episode, the duration of which may last from around ten minutes to a few hours.

Hepatic colic proceeds in accordance with a substantially identical mechanism, principally because of the abrupt tensioning of the biliary tracts, by transient blocking of a stone, either in the cystic channel or in the main biliary tract.

The doctor may prescribe anti-inflammatories in order to reduce the renal tension so that the stone is discharged more easily, or antispasmodics or non-opiate analgesics.

The term dysmenorrhea designates painful periods. These pains precede or accompany the periods.

There are several types of painful period, those is that occur during the first periods, those that occur after several years of periods without pain; pains without organic cause and those that have such causes.

The organic causes may be various.

These are pelvic pains (of the lower abdomen), median, sometimes of the colic type, other times more throbbing and continuous. They appear either a little before the periods or at the start thereof, or towards the end, or manifest throughout the duration thereof.

These pains are of highly variable intensity and may range from a simple nuisance to acute paroxysmal pain.

Hypercontractility of the myometrium is the main cause of these afflictions; however, it has been noted that an endometrial hypersecretion of prostaglandin E2 would appear to be an important factor in essential or functional dysmenorrhea.

When they are very intense, they may be a considerable problem and various symptomatic treatments have been proposed, comprising the administration of analgesics, non-steroidal anti-inflammatories, antispasmodics or oestroprogestatives for example.

Endometriosis is characterised by the presence of abnormal endometrium fragments. In general, when the ovum is not fertilised, the endometrium is eliminated during the periods and renewed subsequently, but when this tissue continues its development outside the uterus it cannot be discharged. The most frequent locations for endometriosis are the ovaries with the formation of cysts, the fallopian tubes, the peritoneum, the region situated between the bladder, the uterus and the vagina and the regions situated between the vagina and the rectum. Depending on the region concerned, digestive endometriosis, rectal endometriosis, pulmonary endometriosis and vesicle endometriosis are spoken of, and deep endometriosis when the lesions affect the wall of the pelvic organs in depth.

Among the clinical signs of endometriosis, low abdominal pains and bleeding of genital origin are frequent. The chronic pelvic pains are located at the lower abdomen and also affect the leg or the lower back. The presence of severe pelvic pains that have persisted for several weeks or months may evoke the presence of an endometriosis. Loss of consciousness may also be found in the case of strong pain. Endometriosis causes severe pain during the periods, which may be haemorrhagic. The presence of pains preceding the periods is also observed.

These manifestations correspond to dysmenorrhea of the secondary type. They are found in 75% to 90% of cases of endometriosis. These are pelvic pains conventionally present just before the start of the periods and persistent for 12 to 72 hours. The pains are often described as intermittent, precise, spasmodic, irradiating towards the buttocks and thighs. They are essentially explained by the uterosacral and rectovaginal location. They are timed by the hormonal variations of the cycle.

The presence of severe dysmenorrhea evokes secondary endometriosis, in particular in the case of rectal endometriosis in which the dysmenorrhea appears to be more severe and more lengthy.

Menstrual pain is an expression of tissue hypoxia following hypercontractility of the myometrium and to the vasoconstriction of its arterioles. Three families of uterotonic and vasoconstricting agents have a recognised responsibility for this problem: prostaglandins, the role of which is preponderant in 80% to 90% of cases, vasopressin arginin and leukotrienes.

The endometrial hypersecretion of prostaglandin would appear to be the main etiological factor in essential or functional dysmenorrhea. It therefore appears clear that an excess of prostaglandins is the physiological denominator common to all functional or organic dysmenorrheas. The treatment of endometriosis and associated dysmenorrheas often consists of the administration of progestatives and/or anti-inflammatories. However, it has been found that at least 30% of women are not relieved by anti-inflammatories taken alone. It may be thought that the role of anti-inflammatories such as ibuprofen in the reduction of menstrual flow contributes to the non-discharge of abnormally situated endometrium and does not afford relief. In the case of these pains, it would therefore be necessary to limit this contribution, which is however used in the treatment of pain in general.

Because of the suddenness and/or intensity of pain associated with all these pathologies, it is very important for a drug that is administered to act as quickly as possible and to relieve this pain in its various components.

With regard to tablets or capsules comprising an active principle in dry and compacted form, it is necessary for it to pass through a step of disaggregation and then of dissolution before it can be absorbed gastrointestinally.

In fact, for these galenic forms, the action time is around fifteen minutes.

Lyophilised forms have been developed. However, this is not possible for all active principles; the manufacturing method is particularly expensive and the forms are very friable and therefore sensitive to being handled.

Liquid forms in the form of a solution or suspension that can be drunk, in which the active principle is dissolved, have been proposed. However, these forms are difficult to transport and furthermore they require a sometimes special formulation involving the use of flavours or agent for masking the taste and of a preservative—this is because a large number of active principles, in particular non-steroidal anti-inflammatories such as ketoprofen or ibuprofen (derived from aryl-propionic acid) are bitter. Such compounds also cause stomach burns via their inhibiting effect on Cox. The effervescent forms to be diluted extemporaneously also often have this drawback, as well as the intrinsic unpleasant/bitter taste of the active principle.

Soft capsules filled with concentrated solutions or suspensions have also being proposed.

The associated technology is particularly expensive and the nature of the shell (mainly soft gelatine) makes them sensitive in an aggressive environment, in particular when the storage and handling temperatures are high.

In the case of the forms consisting of soft gelatine capsules filled with a solution or suspension of ibuprofen for example, the formation of impurities (esters and diesters) has been found, through the esterification of the ibuprofen molecule (which, being a derivative of aryl-propionic acid, comprises a carboxylic acid function) with the alcohol functions of the polyethylene glycol or sorbitol used in the solvent filling the capsules, and this as from a temperature of 25° C.

Thus, although ibuprofen and the other derivatives of propionic acid have numerous advantages in the treatment of pain, the use thereof for treating the aforementioned pathologies presents challenges to be noted, such as those mentioned above, knowing that furthermore stomach burns are often found when taking them.

Antispasmodics act at the smooth muscular fibres of the digestive tract, the urinary tracts and the uterine muscle. They are generally indicated in particular in hepatic and renal colic, spasmodic and painful manifestations, and the biliary, urinary and uterine tracts.

In particular, phloroglucinol (1,3,5-benzenetriol) is musculotropic antispasmodic in the well-known family of triphenols. It is frequency prescribed to attenuate spasms in digestive functional disorders (colitis), renal or hepatic colics and certain gynaecological pains.

Thus there really exists a need for galenic forms intended for the treatment of acute pains such as those indicated above, namely rapid-action galenic forms, the active principles of which are protected from degradation over time under ambient temperature conditions, easy and practical to transport and to use, inexpensive to manufacture, and the action of which in the treatment of the pains mentioned above is maximum/optimum.

It is not impossible to associate various active principles belonging to various therapeutic classes sometimes prescribed by doctors via doses spread out over time.

However, the association of a plurality of active principles in the same pharmacological composition further complicates and accentuates the problems mentioned above. Thus, whether it be a case of the organoleptic properties, chemical stability, the manufacturing method, or the secondary effects of one active principle vis-à-vis the second, increasing the number of active principles of a different chemical nature to be combined merely makes the task more complex and efficacy may be impaired.

More particularly with anti-inflammatories derived from aryl-proprionic acid (such as ketoprofen or ibuprofen for example), because of the propensity of these molecules to esterify and to form impurities, association with active principles containing alcohol functions increases the problems, even more so if the specialities comprising them are subjected to temperature conditions facilitating degradation and/or the formation of impurities.

The inventors propose a pharmaceutical preparation that makes it possible to solve all the problems mentioned above and to relieve the pains indicated above quickly and effectively.

The advantages, in some cases unexpected and surprising, of the pharmaceutical composition according to the invention will emerge from the following detailed description.

The present invention thus relates to an anhydrous pharmaceutical composition comprising an anti-inflammatory principle chosen from the derivatives of aryl-propionic acid, an antispasmodic in the triphenol family and an effervescent excipient.

Anhydrous, within the meaning of the present invention, means that the pharmaceutical composition is characterised in that it contains only very little water, in particular less than 2% by mass, more particularly less than 1% by mass. When reference is made to the expression anhydrous with regard to the components of the effervescent excipient base (citric acid or sodium bicarbonate for example), it is meant in this case that these compounds do not contain any crystallisation water.

In one embodiment, the composition is in the form of a single dose.

In a particular embodiment, the composition according to the invention is buffered.

In a particular embodiment, the anti-inflammatory chosen from the derivatives of aryl-proprionic is chosen from the group consisting of ketoprofen, dexketoprofen, naproxen, ibuprofen and flurbiprofen.

Even more particularly, the anti-inflammatory is ketoprofen or ibuprofen, and more particularly still ibuprofen.

In a particular embodiment of the invention, the musculotropic antispasmodic of the triphenol family is 1,3,5-benzenetriol.

The effervescent excipient can be chosen from the associations comprising at least one organic acid (and/or an organic-acid salt) with at least one base (or base salt).

In the context of a preferred embodiment of the invention, said effervescent excipient comprises the association of at least one organic acid and/or at least one organic-acid salt with at least one strong base and/or at least one salt of a strong base. In the context of this preferred embodiment, said organic acid is advantageously chosen from the group consisting of citric acid, tartaric acid, malic acid, lactic acid, acetic acid, glutaric acid, benzoic acid and adipic acid. Said base acts in the form of sodium bicarbonate, sodium carbonate, calcium carbonate, magnesium carbonate, sodium hydroxide, potassium hydroxide, potassium bicarbonate or potassium carbonate.

In a particular advantageous and preferred fashion, the pharmaceutical compositions of the invention consist of anhydrous effervescent solid galenic forms, especially buffered. They are in particular in the form of effervescent tablets, effervescent granules or buffered effervescent powders. The effervescent excipient is generally and opportunely responsible for a buffer effect and effervescence.

Preferably, the organic acid, in particular citric acid, or the salt thereof is in anhydrous form.

The carbonate or bicarbonate is also preferentially in anhydrous form.

As indicated above, the preferred pharmaceutical form according to the invention is buffered. This means that the aqueous solutions obtained by dissolution of a pharmaceutical form according to the invention have a pH of between 4 and 7, particularly between 5 and 7, more particular still around 6, and these solutions constitute buffer solutions the pH of which varies only a little when strong acid it added. This is particularly advantageous in the case of oral administration, in that, once in the stomach, the pH of the solution is only little influenced by the acidity of the stomach environment. In a particular and especially preferred manner, the same system is generally and opportunely responsible for the buffer effect sought and the effervescence.

The preferred excipient for conferring effervescence and the buffer effect sought can be represented by the pair: citric acid+sodium bicarbonate, preferably in anhydrous form in both cases.

Thus, in a particular embodiment of the invention, the effervescent excipient comprises, or consists of, the association of citric acid+sodium bicarbonate.

A disintegrating agent facilitating rapid effervescence may be included in the pharmaceutical form, in particular in the effervescent excipient. Thus the grains are less compact and less contiguous, which contributes to the more rapid insinuation of water and a more rapid effervescence and therefore a more rapid dissolution of the composition and the active principles contained.

It is in fact important for the pharmaceutical form according to the invention to be in the anhydrous form. This is because any hydration before use thereof would risk causing degradation of the active principles and the formation of impurities, for example via esterification of the acid function of the anti-inflammatory, altering the composition and thus causing a drop in efficacy.

Whatever its exact form, the medicinal association corresponding to the pharmaceutical composition according to the invention generally contains from 50 to 220 mg of 1,3,5-benzenetriol. It contains in particular 50, 60, 70, 80, 100, 120, 150, 160, 180, 200 or 220 mg of 1, 3, 5-benzenetriol. Preferentially between 80 mg and 160 mg.

In the same way, whatever its exact form, the medicinal association corresponding to the pharmaceutical composition according to the invention generally contains from 50 to 500 mg of an anti-inflammatory chosen from the derivatives of aryl-propionic acid. The quantity varies according to the nature of the anti-inflammatory chosen.

According to a preferred variant, the composition according to the invention comprises 50 to 400 mg of ibuprofen. Preferentially 200 mg to 400 mg.

According to a preferred variant, the composition according to the invention comprises 50 to 200 mg of ketoprofen. Preferentially 100 to 150 mg.

According to a preferred variant, the composition according to the invention comprises 200 to 1000 mg of naproxen. Preferentially 500 to 1000 mg, particularly again 750 mg to 1000 mg.

According to a preferred variant, the composition according to the invention comprises 100 to 400 mg of ibuprofen and 50 to 160 mg of 1,3,5-benzenetriol. Preferentially 200 of ibuprofen and 80 mg of 1,3,5-benzenetriol or 400 mg of ibuprofen and 160 mg of 1,3,5-benzenetriol.

The pharmaceutical compositions of the invention are especially used for treating spasmodic disorders, such as spasmodic colites, hepatic colics, renal colics and dysmenorrheas, in particular orally.

More particularly the invention relates to a pharmaceutical composition according to the invention for use thereof in the treatment of secondary dysmenorrheas associated with an endometriosis, particularly orally.

The endometriosis may be a digestive endometriosis, a rectal endometriosis, a pulmonary endometriosis, a vesical endometriosis or a deep endometriosis, particularly orally.

Thus the invention also relates to a pharmaceutical composition according to the invention for use thereof in the treatment of secondary dysmenorrheas associated with a rectal endometriosis, particularly orally.

Thus the invention also relates to a pharmaceutical composition according to the invention for use thereof in the treatment of secondary dysmenorrheas associated with a pulmonary endometriosis, particularly orally.

Thus the invention also relates to a pharmaceutical composition according to the invention for use thereof in the treatment of secondary dysmenorrheas associated with a vesicle endometriosis, particularly orally.

Thus the invention also relates to a pharmaceutical composition according to the invention for use thereof in the treatment of secondary dysmenorrheas associated with a digestive endometriosis, particularly orally.

Thus the invention also relates to a pharmaceutical composition according to the invention for use thereof in the treatment of secondary dysmenorrheas associated with a deep endometriosis, particularly orally.

The invention thus relates to a composition according to the invention for use thereof in the treatment of spasmodic colitis orally.

The invention thus relates to a composition according to the invention for use thereof in the treatment of hepatic colics orally.

The invention thus relates to a composition according to the invention for use thereof in the treatment of renal colics, in particular orally.

The invention thus relates to a composition according to the invention for use thereof in the treatment of renal colic crisis, in particular orally.

The invention relates more particularly to a composition according to the invention for use thereof in maintenance treatment in the 24 to 48 hours following the renal colic crisis, orally.

The invention thus relates to a composition according to the invention for use thereof in the treatment of dysmenorrheas orally.

The pharmaceutical compositions according to the invention can be prepared by direct compression or by wet granulation during which it will be sought to prevent the contact of the water with the 1,3,5-benzenetriol, to avoid any oxidation of this compound, followed by compression/tableting.

In particular, the present invention also relates to a method for preparing a composition according to invention and comprises the following steps:

a) preparation of an effervescent mixture comprising an association of at least one organic acid and/or at least one organic acid salt with at least one strong base and/or at least one salt of a base and optionally a pharmaceutical excipient such as a taste corrector or a disintegrating agent,

b) addition of an aqueous or hydroethanolic solvent and optionally a surfactant pharmaceutical excipient,

c) granulation,

d) drying and optionally calibration in order to obtain an effervescent mixture suited to the compression operation,

e) preparation of a preliminary mixture containing the anti-inflammatory principle chosen from the derivatives of aryl-propionic acid, in particular ibuprofen, and an antispasmodic in the triphenol family, in particular 1,3,5-benzenetriol, and optionally taste-correcting excipients and lubricants,

f) addition of the dry effervescent mixture obtained at d),

g) homogenisation and optionally sieving of the whole obtained in order to obtain an effervescent composition.

In a particular embodiment, the powdery composition obtained at g) can be tableted by compression in order to obtain tablets in accordance with techniques known to persons skilled in the art.

The granulation step provides for the addition of an aqueous or hydroethanolic solvent that enables the mixture to be granulated. When water is used an aqueous solvent, it will preferentially be demineralised water.

Hydroethanolic means that the proportion of water by volume is between 10% and 50%, preferentially between 20% and 30%, more particularly 25%, and the proportion of ethanol by volume is between 50% and 90%, more particularly between 70% and 80%, and more particularly again approximately 75%.

The hydroethanolic or aqueous granulation solvent is added to effect the granulation in a quantity of approximately 0.2% to 3%, particularly 0.2% to 1%, more particularly again 0.3% to 1%, by mass, with respect to the mass of the effervescent mixture of step a).

A weak effervescent reaction is triggered during this external addition of 0.5% to 10% by weight of hydroalcoholic solvent, and will then continue by itself since it generates water; it will be quickly blocked by the drying—which can be done under vacuum—that follows the granulation.

The granulation and drying steps serve for producing the effervescent excipient mixture, to which there are added, in a subsequent operation, the active principles and optionally one or more lubricating or binding excipients; by way of example, a granulation is carried out by wet method on three compounds: sodium bicarbonate, citric acid and optionally glycine (as a binder).

In a particularly effective manner, the manufacturing method uses vacuum during the drying. This technique shortens the manufacturing time, thus improving productivity and saving on energy without forgetting the lower risk of chemical destabilisation by treatment using solvents.

The granulation and drying device of the aforementioned steps comprises mixing means comprising for example a mechanical agitation system with blades for destroying clumps, means for introducing granulation solvent, comprising a system for aspirating granulation solvent by creating a vacuum or a spray system supplied by pump, and optionally temperature-regulation means comprising a double wall where a heat-transfer fluid circulates, and means for creating a vacuum.

Thus the raw materials are mixed in the granulation/drying device and then said granulation solvent is introduced into this granulation/drying device, agitation of all the raw materials and the granulation solvent is carried out at atmospheric pressure until granulation is obtained, and then said drying of the granules obtained is carried out, preferentially under vacuum.

Such a method makes it possible to avoid any contact of the triphenol, in particular 1,3,5-benzenetriol, with water since this contact may cause oxidation of triphenol and the appearance of degradation products.

The effervescent mixture is thereby dried to a very low residual moisture of around less than 2%, preferentially less than 1%.

The mean granulometry of the grains of the effervescent excipient mixture is thus between 250 and 750 μm, more particularly around 500 μm, 100% of the grains being less than 1000 μm.

The effervescent excipient mixture is subsequently mixed with the powdery active principle association and the whole is homogenised in order to obtain a final effervescent powdery mixture representing the composition according to the invention.

This final composition may comprise a ratio by mass of active principles to effervescent excipient of between ½ and ¼, and particularly ⅓.

This mixture can be used as such, packaged in the form of a single dose in sticks or tablets in order to obtain tablets with a mass ranging from 2 to 10 g, in particular 3 to 5 g, for example.

The composition according to the invention may also be in the form of effervescent tablets obtained by direct compression.

In this case, all the ingredients of the composition, namely the effervescent excipient and the active principles, are mixed together, a direct compression excipient is added and the whole is homogenised and the assembly is compressed on a tablet press.

Such a method avoids any use of water and any trace of water that could contribute to degradation of the phloroglucinol.

Because of its excellent compressibility, cellulose is one of the diluents/binders most used in the formulations for direct compression.

The tablets may thus be obtained by direct compression of the mixture containing the effervescent excipient, the active principles and the direct compression excipient via a uniaxial press, an alternating compressing machine or a rotary compressing machine for example.

Furthermore, many unexpected advantages have been noted for the compositions according to the invention.

Firstly, the association of the two active principles—ibuprofen and 1,3,5-benzenetriol—in effervescent form results in a potentialising synergy on pain and more particularly on spasmodic pain. Such synergy makes it possible, for the same therapeutic effect, to reduce the quantity of active principle administered. It is clear that, in the pharmaceutical form according to the invention, the inventors were able to note an effect of synergy of one active principle on the other in relation to spasm and its painful component, including by the acid buffer/base effect of the buffered effervescent pair.

It is indeed the triptych of ibuprofen and 1,3,5-benzenetriol in buffer effervescent form that provides the potentialising synergy observed and which is particularly advantageous in the context of the treatment of the pains indicated in the descriptions.

This is entirely surprising, which makes it possible to envisage an adaptation, i.e. a reduction in the dosage in the case of intolerance, limitation necessary for the daily dose of anti-inflammatory, limitation of the sodium dose for reason of hypertension or gastric pains due to anti-inflammatories or being able to repeat the administration of the composition during painful crises, while avoiding exceeding the maximum doses.

Furthermore, because of the fact that the two active principles are formulated together in an effervescent form, this makes it possible to reduce the quantity of sodium administered in comparison with the separate administration of two effervescent compositions each containing an active principle. This is because the repeated massive administration of an effervescent pharmaceutical composition, via the addition of sodium, contributes to increasing blood pressure. The present invention makes it possible, for the same quantity of the two active principles, to limit the quantity of sodium and is therefore particularly suited to the treatment of patients having or liable to have arterial hypertension.

The invention thus relates to a composition according to the invention for use thereof in the treatment of the pathologies mentioned above such as dysmenorrheas associated with digestive endometriosis, dysmenorrheas associated with rectal endometriosis, dysmenorrheas associated with pulmonary endometriosis, dysmenorrheas associated with vesical endometriosis and with deep endometriosis, the pains associated with hepatic colic or the pains associated with renal colic, in a patient suffering from or liable to suffer from hypertension.

The effervescent form has the advantages of the liquid form, that is to say a rapid action, since the active principles are already in solution when they arrive in the stomach.

Another superiority of the composition according to the invention has also been remarked. As mentioned above, anti-inflammatories such as ibuprofen or ketoprofen are bitter. The inventors have noted a slightly sugary taste of 1,3,5-benzenetriol, which masks the bitterness of the other active principle, without any need for complex and expensive formulation involving the use of flavourings.

Finally, the anhydrous dry form makes the active principles contained particularly inert, which limits or even prevents the formation of impurities.

In fact, during the storage of tablets with a composition according to the invention over a long period, no degradation of the active principles or esterification of the aryl-proprionic acid derivative was noted.

The composition according to the invention may also comprise any other pharmaceutically acceptable excipient. Thus, although not essential, the composition according to the invention may comprise taste correctors, binders, flow regulators and lubricants (silica or magnesium palmitate/stearate), disintegrators (for example polyvinyl pyrrolidone) or other excipients allowing galenic formation or better performance of the manufacturing or compression operations.

### EXAMPLES

### Effervescent Tablet

Quantities for a tablet:

Anhydrous citric acid: approximately 830 mg

Anhydrous sodium bicarbonate: approximately 2250 mg

Taste correctors: approximately 50 mg

Lubricants: approximately 70 mg

Surfactant: approximately 0.75 mg

Wet-granulation method: the ingredients of the effervescent excipient are incorporated successively in a mixer. The premixing takes place in 3 minutes. Wettings carried out by spraying or aspiration of the solvent under partial vacuum, consisting of a hydroethanolic mixture (25% water and 75% ethanol). The quantity of granulation solvent is 0.9% with respect to the weight of the powder.

The granulation is carried out at atmospheric pressure for 5 minutes.

Drying is obtained by reducing the atmospheric pressure to 70 millibar; at this pressure, the boiling point of the solvent is reached. Drying is continued by raising the temperature to a residual moisture content of the powder of less than 0.5%.

The premix containing the active principles is prepared by intimate mixing in quantities corresponding to a ratio of 200 mg of ibuprofen to 80 mg of phloroglucinol.

The two mixtures are homogenised and then compressed by means of a press in a ratio such as to obtain 200 mg of ibuprofen and 80 mg of phloroglucinol in a tablet with a mass of approximately 3.5 g.

### Results of Stability Studies

Ibuprofen-Phloroglucinol 200 mg/80 mg Effervescent Tablets

| | | Results | Results |
|---|---|---|---|
| | Compliance | at | at 9 |
| Determinations | limits | release | months |
| Organoleptic | Effervescent | Compliant | Compliant |
| characteristics | tablets, | | |
| | white to | | |
| | yellowish- | | |
| | white in | | |
| | colour | | |
| Test | | | |
| Disaggregation | <5 min | 1′50″ to | 2′00″ to |
| | | 2′00″ | 2′10″ |
| pH determination | 5.50 to 6.50 | 6.06 | 6.11 |
| Substances related | | | |
| to ibuprofen | | | |
| 4- | 0.20% | ND | ND |
| isobutylacetophenone | | | |
| other impurity | 0.20% | ND | ND |
| Sum of impurities | 0.70% | ND | ND |
| Substances related | | | |
| to phloroglucinol | | | |
| Phloroglucide | 0.20% | ND | 0.04% |
| other impurity | 0.20% | ND | ND |
| Sum of impurities | 1.0% | ND | 0.04% |
| Dosages | | | |
| Ibuprofen per tablet | 0.200 g | 0.197 g | 0.198 g |
| | (0.190 g to | | |
| | 0.210 g) | | |
| Phloroglucinol per | 80 mg | 79.21 mg | 79.54 mg |
| tablet | (76 mg to | | |
| | 84 mg) | | |

ND: not detected.

### Investigation of Impurities in the Active Principles

The active principles ibuprofen and phloroglucinol dihydrate are listed in the 9th edition of the European Pharmacopoeia.

The exclusion, identification and qualification thresholds for impurities in the two active principles are in accordance with ICH Q3 A.

Below is a summary table.

| | | | Reporting | | Identification | | Qualification | |
|---|---|---|---|---|---|---|---|---|
| Active | Mono- | Dose | threshold | | threshold | | threshold | |
| principle | graph | max | ICHQ3 A | Ph. Eur | ICH Q3 A | Ph. Eur | ICH Q3 A | Ph. Eur |
| Ibuprofen | Ph. Eur | ≤2 g/d | 0.05% | 0.03% | 0.1% | 0.05% | 0.15% | 0.15% |
| | 9.0 | | | | | | | |
| Phloroglucinol | Ph. Eur | ≤2 g/d | 0.05% | 0.05% | 0.1% | 0.1% | 0.15% | 0.15% |
| dihydrate | 9.0 | | | | | | | |

Investigation of Impurities in the Finished Product (Ibuprofen+Phloroglucinol 200 mg/80 mg Effervescent Tablets)

The thresholds for exclusion, identification and qualification of impurities in the finished product correspond to ICH Q3 B requirements. Below are summary tables.

The thresholds for exclusion, identification and qualification of impurities in the finished product correspond to ICH Q3 B requirements. Below are summary tables.

| | | Reporting threshold | |
|---|---|---|---|
| | | | Analysis of the |
| Finished | Maximum daily | | finished |
| product | dose | ICH Q3 B | product |
| Ibuprofen | >1 g/d | 0.05% | 0.05% |
| Phloroglucinol | ≤1 g/d | 0.10% | 0.10% |

Identification
threshold

| | | | Analysis of |
|---|---|---|---|
| | | | the |
| Finished | Maximum daily | | finished |
| product | dose | ICH Q3 B | product |
| Ibuprofen | >10 mg-2 g | 0.20% | 0.20% |
| Phloroglucinol | >10 mg-2 g | 0.20% | 0.20% |

These results show that the forms according to the invention preserve the deleterious interactions of the active principles with each other in that no impurities are formed even over 9 months.

Qualification
threshold

| | | | Analysis of |
|---|---|---|---|
| Finished | Maximum daily | | the finished |
| product | dose | ICH Q3 B | product |
| Ibuprofen | >100 mg-2 g | 0.20% | 0.20% |
| Phloroglucinol | >100 mg-2 g | 0.20% | 0.20% |

### Pharmacological Effect

The analgesic and/or antispasmodic effect is assessed by a test in which a painful spasm is induced by the intraperitoneal injection of acetic acid in a mouse.

Three compositions are tested by intragastric administration: solution reconstituted (pH 5) by dissolution in water of an effervescent tablet according to the invention (200 mg ibuprofen+160 mg phloroglucinol), solution reconstituted (pH 6) by dissolution in water of an effervescent tablet according to the invention but comprising only ibuprofen (200 mg), solution reconstituted (pH 5) by dissolution in water of a tablet according to the invention but comprising only phloroglucinol (160 mg).

The dose administered to the mice is 5 times the human dose, that is to say therefore 100 mg/kg with regard to ibuprofen and 80 mg/kg with regard to phloroglucinol.

The inhibiting action of the compositions on pain is assessed on a painful spasm caused in the mouse by the intraperitoneal (IP) injection of a dilute 1% acetic acid solution.

A reference batch of mice receives the distilled water by intragastric method at 20 ml/kg.

One batch of mice receives, by the same method, the composition comprising ibuprofen alone.

One batch of mice receives, by the same method, the composition comprising the phloroglucinol alone.

One batch of mice receives, by the same method, the composition comprising the association of ibuprofen+phloroglucinol.

An injection of 0.2 ml of an acetic acid solution (1% in water) was carried out in the peritoneum of each mouse.

Spasmodic pain syndrome is characterised by twisting movements of the dorso-abdominal musculature.

After the injection of the acetic acid solution and a latency time of 5 minutes, the number of twistings for the following 15 minutes is counted.

The analgesic activity is expressed as a percentage of inhibition of the spasmodic pain crisis for each group treated by the therapeutic compositions in comparison with reference animals treated with distilled water.

| | | Phloroglucinol + |
|---|---|---|
| Phloroglucinol | | ibuprofen |
| alone | Ibuprofen alone | association |
| 35%* | 44%* | 89%*** |

*statistically significant p = 0.05
***statistically significant p = 0.001

It is found that, in all cases, the antispasmodic action is manifested.

However, it is much greater and highly significant for the association. Furthermore, the action of the association (89%) is greater than the sum of the percentages obtained (79%) with the solutions containing a single active principle, which demonstrates a potentialising antispasmodic synergy of this association in this buffered effervescent form. This is because preliminary results obtained with non-effervescent and non-buffered aqueous solutions obtained by simple dissolution of the active principles in water illustrate a lesser analgesic effect as products alone than the association as well as a synergic effect this is equally less (than the one obtained because of the buffered effervescent form according to the invention).

These results are detailed below wherein the products according to the invention (buffered effervescent form) are compared with a formulation of a non-effervescent and non-buffered aqueous solution obtained by simple dissolution of the active principles (ibuprofen and phloroglucinol) at the same dosage, in pure water.

The batches of animals treated according to the following protocols are implemented:

A control batch of mice receives distilled water intragastrically at a rate of 20 ml/kg.

A batch of mice receives by the same route the composition comprising 100 mg/kg of ibuprofen+80 mg/kg of phloroglucinol from an effervescent tablet according to the invention (according to example above) dissolved in distilled water.

A batch of mice receives by the same route the composition comprising 100 mg/kg of ibuprofen+80 mg/kg of phloroglucinol from the suspension of the two active principles in 3% gum water (considering the low solubility of the active principles).

Pharmacological activity was evaluated in the same way as above.

The results are summarized in the table below.

| Ibuprofen + phloroglucinol | Ibuprofen + phloroglucinol |
|---|---|
| effervescent buffered | non-effervescent non- |
| combination | buffered combination |
| 93%* | 59%* |

*statistically significant p = 0.001

Examination of the results shows that regardless of the administration of the product, the pharmacological activity manifests itself and it is nevertheless considerably greater and synergistic in nature since in the case of an effervescent buffered formulation according to the invention the effect is 60% higher compared with the non-buffered and non-effervescent form.

1. Anhydrous pharmaceutical composition comprising an anti-inflammatory principle chosen from the derivatives of aryl-propionic acid, an antispasmodic of a triphenol and a buffered effervescent excipient.

2. Composition according to claim 1, wherein the anti-inflammatory chosen from derivatives of aryl-propionic acid is chosen from the group consisting of ketoprofen, dexketoprofen, naproxen, ibuprofen and flurbiprofen.

3. Composition according to claim 1, wherein that the antispasmodic of a triphenol family is 1,3,5-benzenetriol.

4. Composition according to claim 1, wherein the buffered effervescent excipient comprises an association of at least one organic acid and/or at least one organic acid salt with at least one strong base and/or at least one salt of a strong base.

5. Composition according to claim 1, wherein the effervescent excipient comprises an association of citric acid+sodium bicarbonate.

6. Composition according to claim 1, which is in a form of effervescent tablets, effervescent granules or effervescent powders.

7. A method of orally treating spasmodic disorders chosen from spasmodic colitis, hepatic colic, renal colic and dysmenorrhea which comprises orally administering to a patient in need thereof the composition according to claim 1.

8. The method according to claim 7, wherein said dysmenorrhea is secondary dysmenorrheas associated with an endometriosis.

9. The method according to claim 7, wherein said spasmodic disorder is renal colic crisis.

10. Method for manufacturing a composition according to claim 1, comprising the following steps:
a) preparing an effervescent mixture comprising an association of at least one organic acid and/or at least one organic acid salt with at least one strong base and/or at least one salt of a base,
b) adding an aqueous or hydroethanolic solvent,
c) granulating,
d) drying to form a dry effervescent mixture,
e) preparing a preliminary mixture containing an anti-inflammatory principle chosen from derivatives of aryl-propionic acid and an antispasmodic active principle of a triphenol,
f) adding the dry effervescent mixture obtained at d),
g) homogenizing and obtaining of an effervescent composition.

11. Composition according to claim 2, wherein the antispasmodic of a triphenol is 1,3,5-benzenetriol.

12. Composition according to claim 2, wherein the buffered effervescent excipient comprises an association of at least one organic acid and/or at least one organic acid salt with at least one strong base and/or at least one salt of a strong base.

13. Composition according to claim 3, wherein the buffered effervescent excipient comprises an association of at least one organic acid and/or at least one organic acid salt with at least one strong base and/or at least one salt of a strong base.

14. Composition according to claim 2, wherein the effervescent excipient comprises an association of citric acid+sodium bicarbonate.

15. Composition according to claim 3, wherein the effervescent excipient comprises an association of citric acid+sodium bicarbonate.

16. Composition according to claim 4, wherein the effervescent excipient comprises an association of citric acid+sodium bicarbonate.

17. Composition according to claim 5, wherein the effervescent excipient comprises an association of citric acid+sodium bicarbonate.

18. Composition according to claim 2, which is in the form of effervescent tablets, effervescent granules or effervescent powders.

19. Composition according to claim 3, which is in the form of effervescent tablets, effervescent granules or effervescent powders.

20. Composition according to claim 4, which is in the form of effervescent tablets, effervescent granules or effervescent powders.
